SEQUENCE LISTING (47 sequences)

SEQ ID NO: 1 - protein, 79 aa, Homo sapiens
SEAEDASLLSFMQGYMKHATKTAKDALSSVQESQVAQQARGWVTDGFSSLKDYWSTVKDKFSEFWDLDPEVRPTSAVAA

SEQ ID NO: 2 - protein, 24 aa, Homo sapiens
MQGYMKHATKTAKDALSSVQESQV

SEQ ID NO: 3 - protein, 10 aa, Homo sapiens
MQGYMKHATK

SEQ ID NO: 4 - protein, 32 aa, Homo sapiens
DGFSSLKDYWSTVKDKFSEFWDLDPEVRPTSA

SEQ ID NO: 5 - protein, 21 aa, Homo sapiens
DGFSSLKDYWSTVKDKFSEFW

SEQ ID NO: 6 - protein, 8 aa, Homo sapiens
MQGYMKHA

SEQ ID NO: 7 - protein, 8 aa, Homo sapiens
QGYMKHAT

SEQ ID NO: 8 - protein, 8 aa, Homo sapiens
GYMKHATK

SEQ ID NO: 9 - protein, 8 aa, Homo sapiens
YMKHATKT

SEQ ID NO: 10 - protein, 8 aa, Homo sapiens
MKHATKTA

SEQ ID NO: 11 - protein, 8 aa, Homo sapiens
KHATKTAK

SEQ ID NO: 12 - protein, 8 aa, Homo sapiens
HATKTAKD

SEQ ID NO: 13 - protein, 8 aa, Homo sapiens
ATKTAKDA

SEQ ID NO: 14 - protein, 8 aa, Homo sapiens
TKTAKDAL

SEQ ID NO: 15 - protein, 8 aa, Homo sapiens
KTAKDALS

SEQ ID NO: 16 - protein, 8 aa, Homo sapiens
TAKDALSS

SEQ ID NO: 17 - protein, 8 aa, Homo sapiens
AKDALSSV

SEQ ID NO: 18 - protein, 8 aa, Homo sapiens
KDALSSVQ

SEQ ID NO: 19 - protein, 8 aa, Homo sapiens
DALSSVQE

SEQ ID NO: 20 - protein, 8 aa, Homo sapiens
ALSSVQES

SEQ ID NO: 21 - protein, 8 aa, Homo sapiens
LSSVQESQ

SEQ ID NO: 22 - protein, 8 aa, Homo sapiens
SSVQESQV

SEQ ID NO: 23 - protein, 8 aa, Homo sapiens
DGFSSLKD

SEQ ID NO: 24 - protein, 8 aa, Homo sapiens
GFSSLKDY

SEQ ID NO: 25 - protein, 8 aa, Homo sapiens
FSSLKDYW

SEQ ID NO: 26 - protein, 8 aa, Homo sapiens
SSLKDYWS

SEQ ID NO: 27 - protein, 8 aa, Homo sapiens
SLKDYWST

SEQ ID NO: 28 - protein, 8 aa, Homo sapiens
LKDYWSTV

SEQ ID NO: 29 - protein, 8 aa, Homo sapiens
KDYWSTVK

SEQ ID NO: 30 - protein, 8 aa, Homo sapiens
DYWSTVKD

SEQ ID NO: 31 - protein, 8 aa, Homo sapiens
YWSTVKDK

SEQ ID NO: 32 - protein, 8 aa, Homo sapiens
WSTVKDKF

SEQ ID NO: 33 - protein, 8 aa, Homo sapiens
STVKDKFS

SEQ ID NO: 34 - protein, 8 aa, Homo sapiens
TVKDKFSE

SEQ ID NO: 35 - protein, 8 aa, Homo sapiens
VKDKFSEF

SEQ ID NO: 36 - protein, 8 aa, Homo sapiens
KDKFSEFW

SEQ ID NO: 37 - protein, 8 aa, Homo sapiens
DKFSEFWD

SEQ ID NO: 38 - protein, 8 aa, Homo sapiens
KFSEFWDL

SEQ ID NO: 39 - protein, 8 aa, Homo sapiens
FSEFWDLD

SEQ ID NO: 40 - protein, 8 aa, Homo sapiens
SEFWDLDP

SEQ ID NO: 41 - protein, 8 aa, Homo sapiens
EFWDLDPE

SEQ ID NO: 42 - protein, 8 aa, Homo sapiens
FWDLDPEV

SEQ ID NO: 43 - protein, 8 aa, Homo sapiens
WDLDPEVR

SEQ ID NO: 44 - protein, 8 aa, Homo sapiens
DLDPEVRP

SEQ ID NO: 45 - protein, 8 aa, Homo sapiens
LDPEVRPT

SEQ ID NO: 46 - protein, 8 aa, Homo sapiens
DPEVRPTS

SEQ ID NO: 47 - protein, 8 aa, Homo sapiens
PEVRPTSA

(19) United States
(12) Patent Application Publication
(10) Pub. No.: US 2004/0091496 A1
(43) Pub. Date: May 13, 2004

(54) Vaccine

(76) Inventors: Pascal Mettens, Rixensart (BE); Rene Meykens, Rixensart (BE); Philippe Monteyne, Rixensart (BE)

Correspondence Address: GLAXOSMITHKLINE, Corporate Intellectual Property - UW2220, P.O. Box 1539, King of Prussia, PA, 19406-0939, US

(21) Appl. No.: 10/387,934

(22) Filed: Mar. 13, 2003

(30) Foreign Application Priority Data
Aug. 29, 2002 (WO) PCT/EP02/09650

(51) Int. Cl. A61K 39/00; C12Q 1/68; C07H 21/04; C07K 14/775

(52) U.S. Cl. 424/185.1; 435/6; 435/69.1; 435/320.1; 435/325; 530/359; 536/23.5

(57) ABSTRACT

The present invention relates to novel vaccine therapies, and prophylactic treatments of atherosclerotic diseases. Accordingly there is provided, immunogens comprising specific fragments or derivatives of oxidised Apolipoprotein C-III (ApoCIII). The vaccines of the present invention, comprising said immunogens, are potent in the prevention, or reduction, of atherosclerotic plaque formation over prolonged periods of time, thereby reducing the potential of atherosclerosis leading to coronary or cerebrovascular disease. Also provided are methods of treating or preventing atherosclerosis by active vaccination, or passive vaccination through administration to a patient of an antibody that is capable of binding to the specific fragments of ApoCIII. Specific monoclonal antibodies and their use in therapy of atherosclerosis is provided. There is further provided the use of the immunogens of the present invention in medicine, and methods of their production. The fragments of ApoCIII which form the basis of the immunogens of the present invention, and also the targets for passive immunotherapy, are encompassed within the regions between amino acid numbers 45-76 and, particularly, 12-35 of the mature form of human ApoCIII.

The present invention relates to novel vaccine therapies, and prophylactic treatments of atherosclerotic diseases. Accordingly there is provided, immunogens capable of inducing an immune response against modified Apolipoprotein C-III (ApoCIII) and modified epitopes thereof. The vaccines of the present invention, comprising said immunogens, are potent in the prevention, or reduction, of atherosclerotic plaque formation over prolonged periods of time, thereby reducing the potential of atherosclerosis leading to coronary or cerebrovascular disease. Also provided are methods of treating or preventing atherosclerosis by passive vaccination through administration to a patient of an antibody that is capable of binding to modified ApoCIII and modified epitopes thereof. Specific monoclonal antibodies and their use in therapy of atherosclerosis are provided. There is further provided the use of the immunogens of the present invention in medicine, and methods of their production. Atherosclerosis is the leading cause of death and disability in the developed world, and is the major cause of coronary and cerebrovascular deaths, with approximately 7.2 and 4.6 million deaths per year worldwide respectively (Atherosclerosis is generally described in Harrison's Principles of Internal Medicine (14^{th} Edition, McGraw Hill, p1345-1352), Berliner, J. et al., 1995, Circulation, 91:2488-2496; Ross, R., 1993; Nature, 362:801). The name in Greek refers to the thickening (sclerosis) of the arterial intima and accumulation of lipid (athere) in lesions.

Although many generalised or systemic risk factors predispose to its development, such as a high cholesterol diet and smoking, this disease may affect different distinct regions of the circulation. For example, atherosclerosis of the coronary arteries commonly causes angina pectoris and myocardial infarction. Whilst, atherosclerosis of the arteries supplying the central nervous system frequently provokes transient cerebral ischemia and strokes. In the peripheral circulation, atherosclerosis can cause intermittent claudication and gangrene and can jeopardise limb viability. Involvement of the splanchnic circulation can cause mesenteric ischemia and bowel infarction. Atherosclerosis can affect the kidney directly (eg causing renal artery stenosis), and in addition, the kidney is a frequent site of atheroembolic disease.

Atherogenesis in humans typically occurs over many years, usually many decades. The slow build up of atherogenic plaques in the lining of the vasculature can lead to chronic clinical expressions through blood flow restriction (such as stable effort-induced angina pectoris or predictable and reproducible intermittent claudication). Alternatively, a much more dramatic acute clinical event, such as a myocardial infarction or cerebrovascular accident can occur after plaque rupture. The way in which atherosclerosis affects an arterial segment also varies, an additional feature of the heterogeneity and complexity of this disease. Atheromas are usually thought of as stenotic lesions, or plaques, which can lirmit blood flow, however, atherosclerosis can also cause ectasia and development of aneurysmal disease with an increase in lumen caliber. This expression of atherosclerosis frequently occurs in the aorta, creating a predisposition to rupture or dissection rather than to stenosis or occlusion.

The genesis of atherogenic plaques has been studied in depth. In normal human adults, the intimal layer of arteries contains some resident smooth muscle cells embedded in extracellular matrix and is covered with a monolayer of vascular endothelial cells. Initial stages of atherogenesis involve the development of “fatty streaks” in the walls of the blood vessel resulting from accumulation and deposit of lipoproteins in regions of the intimal layer of the artery. Low-density lipoprotein (LDL) particles, rich in cholesterol, is an example of an atherogenic lipoprotein which is capable of deposition in the vessel walls to form such fatty streaks.

Once deposited within the vessel wall, the lipoprotein particles undergo chemical modification, including both oxidation and non-enzymatic glycation. These oxidised and glycated lipoproteins then contribute to many of the subsequent events of lesion development. The chemical modifications attract macrophages within the vessel walls, which internalise the oxidised LDL and become foam cells which initiate lesions called plaques. It is the atherosclerotic plaques which are responsible for the clinical manifestations of atherosclerosis, either they limit blood flow, or allow aneurism, or may even rupture provoking the coronary or cerebrovascular attacks.

The development of atherosclerosis is a long process which may occur over decades, which is initiated by an imbalance between atherogenic and protective lipoproteins. For example, cholesterol associated with high-density lipoproteins or HDL (so called “good” cholesterol) and low-density lipoproteins or LDL (so called “bad” cholesterol) levels in the circulation are thought to be markers of increased probability of atherosclerosis (Harrison's Principles of Internal Medicine (14^{th} Edition, McGraw Hill, p1345-1352)).

Cholesterol, cholesterol esters, triacylglycerols and other lipids are transported in body fluids by a series of lipoproteins classified according to their increasing density: chylomicrons, Very Low, Low, Intermediate and High density lipoproteins (CM, VLDL, LDL, IDL and HDL respectively). These lipoprotein-complexes consist of a core of hydrophobic lipids surrounded by polar lipids and then by a shell of Apolipoproteins. Currently, there are at least twelve types of apolipoproteins known, A-I, A-II, A-IV, A-V, B, CI, CII, CIII, D, E, H and J. There are at least two functions of these apolipoproteins which are common to all lipoprotein complexes, first they are responsible for the solubilisation of the hydrophobic lipid cores that they carry, and second they are also involved in the regulation of cholesterol lipoprotein uptake by specific cells. The different types of lipoproteins may have different functions, for example LDL (which are rich in cholesterol esters) are thought to be associated with the transport of cholesterol to peripheral tissues for new membrane synthesis.

One of these apolipoproteins, apolipoprotein C-III (ApoCIII), is a 79 amino acid protein produced in the liver and intestine (Brewer et al., J. Biol. Chem. (1974), 249: 4975-4984; Protter, A. A., et al., 1984, DNA, 3:449-456; Fruchart, J. C. et al, 1996, Drugs Affecting Lipid Metabolism, (Eds. Gotto, A. M. et al.), Kluwer Academic Publishers and Fordazione Giovanni Lorenzini, Netherlands, p631-638; Claveny, V. et al., Arteriosclerosis, Thrombosis and Vascular Biology, 15, 7, 963971; U.S. Pat. No. 4,801,531; McConathy, W. J. et al. 1992, Journal of Lipid Research, 33, 995-1003). Apo CIII is a component of CM, VLDL, LDL (Lenich et al., C., J. Lip. Res. (1988) 29, 755-764), and also HDL, and exists as three isoforms: apo CIII0, apo CIII1 and apo CIII2. Apo CIII0 is not glycosylated, however apo CIII1 and apo CIII2 are glycosylated and have respectively one and two sialic acid residues (Ito et al., 1989 J.lipd. Res. Nov 30:11 1781-1787). The sugar moiety consists of disaccharide β-D galactosyl (1-3) α-N-Acetyl-D-Galactosamine attached to threonine 74 of protein chain by O-glycosidic binding (Assman et al., 1989, BBA 541:234-240). In human normolipidemic plasma apo CIII0, apo CIII1 and apo CIII2 represent 14%, 59% and 27% of total apo CIII respectively. Mutagenesis of the glycosylation site of human apo CIII does not affect its secretion and lipid binding (Roghani et al., 1988 JBC 34:17925-32).

Mature Human ApoCIII has the following amino acid sequence: ₁SEAEDASLLSFMQGYMKHATKTAKDALSSVQESQVAQQARGWVTDGFSS LKDYWSTVKDKFSEFWDLDPEVRPTSAVAA₇₉ (SEQ ID.NO. 1).

Plasma concentration of apo CIII is positively correlated with levels of plasma triglycerides (Schonfeld et al., Metabolism (1979) 28: 1001-1010; Kaslyap et al., J. Lip. Res. (1981) 22: 800-810). Liver perfusion studies demonstrate that apo. CIII inhibits the hepatic uptake of triglyceride-rich lipoproteins (TRL) and their remnants (Shelburne et al., J. Clin. Inves., (1980) 65: 652-658, Windler et al., J. Lip. Res. (1985) 26: 556-563). Also in vitro experiments show that apo CIII inhibit the activity of both lipoprotein lipase (LPL) and hepatic lipase (Brown and Bakinsky, Biochim. Biophs. Acta. (1972) 46: 375-382; Krauss et al., Circ. Res. (1973) 33: 403-411; Wang et al., J. Clin. Inves. (1985) 75: 384-390; Mc Conathy et al., J. Lip. Res. (1972) 33: 995-1003; Kinnemen and Enholm, FEBS (1976) 65: 354-357). Moreover, ApoCIII is said to be involved in inhibition of LDL binding to LDL receptors (Fruchart et al. supra), via ApoB.

The role of apo CIII in plasma TRL metabolism has been more defined by the results of recent studies in transgenic animals (Aalto-Setälä et al., J. Clin. Invest. (1992) 90:5 1889-1900.). Plasma accumulation of TRL in mice overexpressing apo CIII has been shown to be associated with reduced plasma VLDL and chylomicron clearance (Harrold et al., J. Lip. Res. (1996) 37 : 754-760) also the inhibitory effect of C apolipoproteins on the LDL receptor of apo B-containing lipoproteins was demonstrated (Clavey et al., Arth. Thromb. and Vase. Biol. (1995) 15 : 963-971).

Previous vaccines in the field of immunotherapy of atherosclerosis have focused on the use of cholesterol as an immunogen to reduce serum cholesterol levels (Bailey, J. M. et al., 1994, Biochemical Society Transactions, 22, 433S; Alving, C. and Swartz, G. M., 1991, Crit. Rev. Immunol., 10, 441-453; Alving, C. and Wassef, N. M., 1999, Immunology Today, 20, 8, 362-366). Others have attempted to alter the activity of the Cholesterol Ester Transfer Protein (CETP) by vaccination (WO 99/15655). Alternatively, some authors have described vaccines using oxidised LDL as the immunogen, in order to inhibit plaque formation after balloon injury in hypercholesterolemic rabbits (Nilsson, J. et al., 1997, JACC, 30, 7, 1886-1891).

Oxidised LDL is implicated in atherosclerosis; it is thought that oxidised LDL accumulates in atherosclerotic lesions. A natural humoral response has been found to occur against oxidised LDL, with the antibodies forming this response having a predominantly IgM isotype, typical of T-cell independent immunity. A memory immune response, indicative of T-cell dependent immunity, is not typically observed.

Active immunotherapy according to the present invention may trigger a humoral response to Apo CIII which is predominantly T-cell mediated. Without wishing to be bound by theory, IgG and/or IgM antibodies may effect a strong response enabling any circulating ApoCIII, preferably oxidised ApoCIII, to be removed from the circulation, whilst reducing the likelihood of generating potentially harmful autoreactive antibodies. This may prevent the development of “fatty streaks” in the walls of the blood vessel which result from accumulation and depositition of lipoproteins in regions of the intimal layer of the artery. As a result this may prevent formation of the foam cells which initiate lesions called plaques.

Immunisation with isolated, modified ApoCIII is provided by the present invention. Immunisation with modified ApoCIII results in a polyclonal IgG response. Immunisations with modified ApoCIII molecule forms a strong immune response to its accessible epitopes and leads to effective generation of a memory immune response.

Immunisation with selected epitopes of modified ApoCIII is also provided by the present invention. Immunisation with selected modified epitopes results in antibodies being generated to fewer epitopes, although the response is still polyclonal. This is advantageous because this minimises the development of autoreactive antibodies, and it can focus the immune response to “non-self” oxidised ApoCIII epitopes in circulating oxidised, or otherwise modified, LDL. In particular, target epitopes include the epitope found within the region between amino acid number 12 and 35, or an epitope found within the region between amino acids 45 and 76 of the human ApoCIII molecule. In addition it is preferred that the immunotherapy targets the epitope that is found within the region between amino acid 12 to 21 or 45 to 65 of human ApoCIII, although further epitopes may also be used.

The sequence of the region between amino acid number 12 and 35 of the human ApoCIII is as follows:

MQGYMKHATKTAKDALSSVQESQV (SEQ ID NO.2).

The sequence of the region between amino acid number 12 and 21 of the human ApoCIII is as follows:

MQGYMKHATK (SEQ ID NO.3)

The sequence of the region between amino acid number 45 and 76 of the human ApoCIII is as follows:

DGFSSLKDYWSTVKDKFSEFWDLDPEVRPTSA (SEQ ID NO: 4)

The sequence of the region between amino acid number 45 and 65 of the human ApoCIII is as follows:

DGFSSLKDYWSTVKDKFSEFW (SEQ ID NO: 5)

The present invention also provides the following fragments of the above peptides within which contain an epitope of ApoCIII which may be modified and targeted by the active or passive immunotherapies of the present invention:

| Peptide Sequence | SEQ ID NO: |
|---|---|
| MQGYMKHA | 6 |
| QGYMKHAT | 7 |
| GYMKHATK | 8 |
| YMKHATKT | 9 |
| MKHATKTA | 10 |
| KHATKTAK | 11 |
| HATKTAKD | 12 |
| ATKTAKDA | 13 |
| TKTAKDAL | 14 |
| KTAKDALS | 15 |
| TAKDALSS | 16 |
| AKDALSSV | 17 |
| KDALSSVQ | 18 |
| DALSSVQE | 19 |
| ALSSVQES | 20 |
| LSSVQESQ | 21 |
| SSVQESQV | 22 |
| DGFSSLKD | 23 |
| GFSSLKDY | 24 |
| FSSLKDYW | 25 |
| SSLKDYWS | 26 |
| SLKDYWST | 27 |
| LKDYWSTV | 28 |
| KDYWSTVK | 29 |
| DYWSTVKD | 30 |
| YWSTVKDK | 31 |
| WSTVKDKF | 32 |
| STVKDKFS | 33 |
| TVKDKFSE | 34 |
| VKDKFSEF | 35 |
| KDKFSEFW | 36 |
| DKFSEFWD | 37 |
| KFSEFWDL | 38 |
| FSEFWDLD | 39 |
| SEFWDLDP | 40 |
| EFWDLDPE | 41 |
| FWDLDPEV | 42 |
| WDLDPEVR | 43 |
| DLDPEVRP | 44 |
| LDPEVRPT | 45 |
| DPEVRPTS | 46 |
| PEVRPTSA | 47 |

The present invention provides vaccine immunogens effective in the prophylaxis or therapy of atherosclerosis which comprise immunogens that raise an immune response against isolated modified epitopes of ApoCIII, or a modified peptide having a primary sequence of any of SEQ ID NO.s 1-47, in which at least one amino-acid of the peptide sequence is modified. The present invention also provides for methods of treatment of atherosclerosis by the administration of the immunogens of the present invention to individuals in need thereof. Most preferably the immunogens of the invention comprise the epitopes listed in SEQ ID NO: 2, 3, 6-22.

The present invention also provides monoclonal antibodies that are specific for modified epitopes of ApoCIII, including those described in SEQ ID NO.s 1-47. Also provided are methods of treatment of individuals by passive administration of the monoclonal antibodies to the individual.

By modified it is meant that at least one amino acid is modified by, for example, oxidation, nitration, glycosylation, hydrogenation, or any other modification which may cause a conformational change to the 3D protein structure. Combinations of different modifications may also be used. Preferably, more than 1, 2, 3, 4, 5, 6, 7, 8, 9 or 10 amino acids of the sequence may be modified. In one embodiment of the present invention, between one and all amino acids are modified. Preferably, the amino acids are oxidised or glycoxylated. In a preferred embodiment of the present invention, oxidation of ApoCIII, or fragments thereof, is performed by use of myeloperoxidase (MPO). MPO is a naturally occurring enzyme. Without wishing to be bound by theory, the hypothesised mechanism of action of MPO follows: MPO (Fe⁺⁺⁺) may react with H₂O₂ to form a complex of (Fe⁵⁺ MPO and H₂O₂). This complex may then react with Cl⁻ to generate HOCl. This hypochloride (HOCl) may react then with NH₂ groups of amino acids to generate chloramines, or with OH groups of Tyrosine residues to generate chlorotyrosine. These chloramines and chlorotyrosines are oxidized molecules which are preferably targeted in the present invention.

By isolated it is meant that the modified ApoCIII molecule has been altered by the hand of man.

Active Immunotherapy

In the first aspect of the present invention, the immunogens of the present invention are capable of generating immune responses against modified epitopes of ApoCIII, including immune responses that recognise the epitopes SEQ ID NO.s 1-47 in which at least one amino-acid is modified. Accordingly, the immunogens may comprise or contain SEQ ID NO's. 1-47 in which at least one amino-acid is modified, or they may comprise or contain synthetic peptides having the sequences listed in SEQ ID NO.s 1-47 in which at least one amino-acid is modified, or the immunogens may comprise or contain mimotopes thereof which retain the functional activity of being able to induce immune responses that recognise the epitopes listed in SEQ ID NO.s 1-47 in which at least one amino-acid is modified. Most preferably the immunogens of the invention comprise the epitopes listed in SEQ ID NO: 2, 3, 6-22 in which at least one amino-acid is modified.

Most preferably the antibodies induced by the immunogens of the present invention are functional in the treatment of atherosclerosis, and in a preferred form of the present invention they prevent uptake of oxidised ApoCIII by macrophages, thus reducing the formation of foam cells, and reducing the formation of atherosclerotic plaques.

The immunogen may comprise or contain modified ApoCIII epitopes, or peptides of SEQ ID NO. 1-47 in which at least one amino-acid is modified, or alternatively the immunogen may comprise or contain fragments of the identified peptides of SEQ ID NO 1-47, lacking 1, 2, 3, 5 or 10 amino acids from either or both of the N- or C-termini of the peptides. Alternatively, the immunogen may comprise or contain a peptide which is longer than SEQ ID NO.s 1-47, that contain SEQ ID NO. 1-47 within the longer sequence and in which at least one amino-acid is modified. Preferably, peptides with 1, 2, 3, 5, 10, or 20 amino acids may be added to either or both of the N- or C-termini of the peptides from the native context of the peptides within human mature ApoCIII. Most preferably, in this case, the longer immunogens are less than 80 amino acids in length, more preferably less than 50 amino acids, more preferably less than 40 amino acids and most preferably less than 25 amino acids long. The immunogen may be longer than those described above if it further comprises a carrier molecule fused to the peptides of the invention as described below.

In yet another alternative, the immunogen may be a true mimotope of the linear sequences described in SEQ ID NO.1-47, or parts or fragments thereof, in which at least one amino-acid is modified, in that the sequence of the peptide mimotope is not-necessarily related to the sequences but may represent a three dimensional conformational epitope which binds to the region corresponding to the folded tertiary structure of modified ApoCIII which is made up of the amino acids of SEQ ID NO.s 1-47, or parts or fragments thereof.

The immunogens of the present invention may, therefore, comprise or contain the peptides encompassing the modified apolipoprotein epitopes themselves, and any mimotope thereof. The meaning of mimotope is defined as an entity which is sufficiently similar to the apolipoprotein epitope so as to be capable of being recognised by antibodies which recognise the apolipoprotein; (Gheysen, H. M., et al., 1986, Synthetic peptides as antigens. Wiley, Chichester, Ciba foundation symposium 119, p130-149; Gheysen, H. M., 1986, Molecular Immunology, 23,7, 709-715); or are capable of raising antibodies, when coupled to a suitable carrier, which antibodies cross-react with the native apolipoprotein.

Peptide mimotopes of the above-identified modified ApoCIII peptides/epitopes may be designed for a particular purpose by addition, deletion or substitution of elected (1, 2, 3, 4, 5 or more) amino acids. Thus, the modified peptides of the present invention may be altered for the purposes of ease of conjugation to a protein carrier. For example, it may be desirable for some chemical conjugation methods to include a terminal (N-and/or C-) cysteine to the apolipoprotein epitope. In addition it may be desirable for peptides conjugated to a protein carrier to include a hydrophobic terminus distal from the conjugated terminus of the peptide, such that the free unconjugated end of the peptide remains associated with the surface of the carrier protein. This reduces the conformational degrees of freedom of the peptide, and thus increases the probability that the peptide is presented in a conformation which most closely resembles that of the apolipoprotein peptide as found in the context of the whole apolipoprotein. For example, the peptides may be altered to have an N-terminal cysteine and a C-terminal hydrophobic amidated tail. Conformational restriction may also take place if N- and C-termini of the peptides are Cysteine residues which may be induced to form a cyclised peptide through a disulphide bond (optionally having additional terminal amino acids for conjugation to a carrier molecule). D and K residues may also be included at N- and C-termini of the peptides of the invention, respectively (or vice versa), in order to form cyclised peptides via a β-lactam bond which can be straightforwardly made between D and K residues (optionally such peptides may have an additional terminal amino acid [such as a Cysteine] for conjugation to a carrier molecule. Alternatively, the addition or substitution of a D-stereoisomer form of one or more of the amino acids may be performed to create a beneficial derivative, for example to enhance stability of the peptide. Those skilled in the art will realise that such altered peptides, or mimotopes, could be a wholly or partly non-peptide mimotope wherein the constituent residues are not necessarily confined to the 20 naturally occurring amino acids. In addition, these may be cyclised by techniques known in the art to constrain the peptide into a conformation that closely resembles its shape when the peptide sequence is in the context of the whole apolipoprotein (for example by the addition of a cysteine at the terminal regions of the peptide to form a disulphide bridge).

The peptide mimotopes may also be retro sequences of the natural apolipoprotein peptide sequences, in that the sequence orientation is reversed; or alternatively the sequences may be entirely or at least in part comprised of D-stereo isomer amino acids (inverso sequences). Also, the peptide sequences may be retro-inverso in character, in that the sequence orientation is reversed and the amino acids are of the D-stereoisomer form. Such retro or retro-inverso peptides have the advantage of being non-self, and as such may overcome problems of self-tolerance in the immune system.

Alternatively, peptide mimotopes may be identified using antibodies which are capable themselves of binding to the modified apolipoprotein, using techniques such as phage display technology (EP 0 552 267 B 1). This technique, generates a large number of peptide sequences which mimic the structure of the native modified peptides and are, therefore, capable of binding to anti-native modified peptide antibodies, but may not necessarily themselves share significant sequence homology to the native apolipoprotein.

In the vaccines of the present invention the epitope or mimotope is preferably linked to a carrier molecule to form an immunogen which enhances the immunogenicity of the epitope. Accordingly, the peptides or mimotopes may be linked via chemical covalent conjugation or by expression of genetically engineered fusion partners, optionally via a linker sequence. The peptides may have two or more Glycine residues as a linker sequence, and often have a terminal exposed cysteine residue for linkage purposes.

The preferred covalent coupling of the epitope of ApoCIII, to the carrier protein can be carried out in a manner well known in the art. Thus, for example, for direct covalent coupling it is possible to utilise a carbodiimide, glutaraldehyde or (N[y-maleimidobutyryloxy]) succinimide ester, utilising common commercially available heterobifunctional linkers such as CDAP and SPDP (using manufacturers instructions). Conveniently, the ApoCIII or fragment thereof is modified before such covalent coupling.

The types of carriers used in the immunogens of the present invention will be readily known to the man skilled in the art. The function of the carrier is to provide cytokine help (or T-cell help) in order to enhance the immune response against the apolipoprotein or apolipoprotein peptide. A non-exhaustive list of carriers which may be used in the present invention include: Keyhole limpet Haemocyanin (KLH), serum albumins such as bovine serum albumin (BSA), inactivated bacterial toxins such as tetanus or diptheria toxins (TT and DT, or the DT derivative CRM197), or recombinant fragments thereof (for example, Domain 1 of Fragment C of TT, or the translocation domain of DT), or the purified protein derivative of tuberculin (PPD). Alternatively the epitopes or may be linked to the carrier in a non-covalent fashion such as association via a liposome carrier or by co-adsorption onto an aluminium salt, which may additionally comprise immunogens capable of providing T-cell help or additional adjuvant immunostimulators. Preferably the ratio of the number of apolipoprotein, or fragment or peptide thereof, to carrier protein is in the order of 1:1 to 20:1, and preferably each carrier should carry between 3-15 apolipoproteins, or peptide or fragment thereof.

In an embodiment of the invention the carrier is Protein D from Haemophilus influenzae (EP 0 594 610 B1). Protein D is an IgD-binding protein from Haemophilus influenzae and has been patented by Forsgren (WO 91/18926, granted EP 0 594 610 B 1). In some circumstances, for example in recombinant immunogen expression systems it may be desirable to use fragments of protein D, for example Protein D ⅓^{rd} (comprising the N-terminal 100-110 amino acids of protein D (WO 99/10375; WO 00/50077)).

Another preferred method of presenting the peptides of the present invention, is in the context of a recombinant fusion molecule. For example, EP 0 421 635 B describes the use of chimeric hepadnavirus core antigen particles to present foreign peptide sequences in a virus-like particle. As such, immunogens of the present invention may comprise modified epitopes of ApoCIII, or epitopes of ApoCIII described in SEQ ID NO.s 1-47, or fragments or mimotopes thereof, in which at least one amino-acid is modified, presented in chimeric particles consisting of hepatitis B core (HepB core) antigen. Additionally, the recombinant fusion proteins may comprise the mimotopes of the present invention and a carrier protein, such as NS1 of the influenza virus. For any recombinantly expressed protein which forms part of the present invention, the nucleic acid which encodes said immunogen also forms an aspect of the present invention. Preferably, the recombinant fusion molecule is made and then modified.

Accordingly, preferred immunogens of the present invention comprise modified epitopes of ApoCIII, or an epitope of SEQ ID NO: 1-47, in which at least one amino-acid is modified, presented in a recombinant expression system (such as HepB core) or conjugated to a carrier protein, such that the recombinant expression system or the carrier protein provide T-cell help for generation of an immune response to SEQ ID NO: 1-47, in which at least one amino-acid is modified, (preferably against modified SEQ ID NO: 2 or 3 for immunogens based on SEQ ID NO: 2, 3, 6-22, and against modified SEQ ID NO: 4 or 5 for immunogens based on SEQ ID NO: 4, 5, 23-47).

In an alternative embodiment of the present invention the immunogenicity of the peptides is enhanced by the addition of T-helper (Th) epitopes. The immunogens of the present invention may, therefore, comprise the peptides as described previously and promiscuous Th epitopes either as chemical or recombinant conjugates or as purely synthetic peptide constructs. The apolipoprotein peptides are preferably joined to the Th epitopes via a spacer (e.g., Gly-Gly) at either the N- or C-terminus of the apolipoprotein peptide. The immunogens may comprise 1 or more promiscuous Th epitopes, and more preferably between 2 to 5 Th epitopes.

A Th epitope is a sequence of amino acids that comprise a Th epitope. A Th epitope can consist of a continuous or discontinuous epitope. Hence not every amino acid of Th is necessarily part of the epitope. Th-epitopes that are promiscuous are highly and broadly reactive in animal and human populations with widely divergent MHC types (Partidos et al. (1991) “Immune Responses in Mice Following Immunization with Chimeric Synthetic Peptides Representing B and T Cell Epitopes of Measles Virus Proteins” J. of Gen. Virol. 72:1293-1299; U.S. Pat. No. 5,759,551). The Th domains that may be used in accordance with the present invention have from about 10 to about 50 amino acids, and preferably from about 10 to about 30 amino acids. When multiple Th epitopes are present, each Th epitope is independently the same or different. Preferably the ratio of the number of modified apolipoproteins, or fragments or peptides thereof, to Th epitope is in the order of 1:1 to 20: 1, and preferably each Th epitope should carry between 3-15 modified apolipoproteins, or peptides or fragments thereof. Th epitopes include as examples, pathogen derived epitopes such as Hepatitis surface or core (peptide 50-69, Ferrari et al., J.Clin.Invest, 1991, 88, 214-222) antigen Th epitopes, Pertussis toxin Th epitopes, tetanus toxin Th epitopes (such as P2 (EP 0 378 881 B1) and P30 (WO 96/34888, WO 95/31480, WO 95/26365), measles virus F protein Th epitopes, Chlamidia trachomatis major outer membrane protein Th epitopes (such as P11, Stagg et al., Immunology, 1993, 79, 1-9), Yersinia invasin and diptheria toxin Th epitopes. Other Th epitopes are described in U.S. Pat. No. 5,759,551 and Cease et al., 1987, Proc. Natl. Acad. Sci. 84, 4249VB60137 4253; and Partidos et al., J. Gen.Virol, 1991, 72, 1293-1299; WO 95/26365 and EP 0 752 886 B.

In one embodiment of the invention, the carrier is a tetanus toxin (TT) peptide T-helper epitope. Examples of TT peptides which may be used as carriers in the present invention include the P2 peptide of TT and the P30 epitope of TT. The TT peptide carriers may be linked to modified Apo CIII epitopes (eg modified SEQ ID NO 1) or fragments thereof as described herein. Preferably, the TT peptide carriers are linked to immunogens which comprise or contain the sequence listed in any of SEQ ID NOs: 1-47, in which at least one amino-acid is modified, or extensions or truncations thereof as described herein. The link between the TT peptide carrier and the immunogen may be through the C-terminus or the N-terminus. Alternatively, the TT peptide carrier may be linked between two or more peptides of modified Apo CIII or fragments thereof. Examples of preferred embodiments are listed below. It will be appreciated that the examples are not an exhaustive list; any combination of whole or fragmented modified Apo CIII and TT T-helper epitope peptides which enhance immunogenicity of the modified Apo CIII epitope may be used in the present invention.

(i). modified ApoCIII-P2

=whole ApoCIII (SEQ ID NO 1) linked to P2 (peptide of TT) at C-term, in which at least one amino-acid of the ApoCIII peptide sequence is modified

(ii). P2- modified ApoCIII

=whole ApoCIII (SEQ ID NO 1) linked to P2 (peptide of TT) at N-term, in which at least one amino-acid of the ApoCIII peptide sequence is modified

(iii). modified (12-35)-P2- modified (12-35)

=P2 between two peptides (SEQ ID NO 2) of ApoCIII in which at least one amino-acid of the ApoCIII peptide sequence is modified

(iv). modified (12-35)—P30- modified (12-35)

=P30 (peptide of TT) between two peptides (SEQ ID NO 2) of ApoCIII in which at least one amino-acid of the ApoCIII peptide sequence is modified

(v). modified (45-65)-P2- modified (45-65)

=P2 between two peptides (SEQ ID NO 5) of ApoCIII in which at least one amino-acid of the ApoCIII peptide sequence is modified

(vi). modified (45-65)-P30- modified (45-65)

=P30 between two peptides (SEQ ID NO 5) of ApoCIII in which at least one amino-acid of the ApoCIII peptide sequence is modified

(vii). modified (45-76)-P2- modified (45-76)

=P2 between two peptides (SEQ ID NO 4) of ApoCIII, in which at least one amino-acid of the ApoCIII peptide sequence is modified

(viii). modified (45-76)-P30- modified (45-76)

=P30 between two peptides (SEQ ID NO 4) of ApoCIII, in which at least one amino-acid of the ApoCIII peptide sequence is modified

The immunogens of the present invention are provided for use in medicine, for use in the treatment or prevention of atherosclerosis, and for formulation into immunogenic compositions or vaccines of the present invention.

Another preferred epitope that forms part of the present invention is the peptide found between amino acids 21 and 35 of human ApoCIII, in which at least one amino-acid of the ApoCIII peptide sequence is modified.

The immunogenic compositions and vaccines comprise one or more immunogens of the present invention as previously described, and may advantageously also include an adjuvant. Suitable adjuvants for vaccines of the present invention comprise those adjuvants that are capable of enhancing the antibody responses against the apolipoprotein immunogen. Adjuvants are well known in the art (Vaccine Design—The Subunit and Adjuvant Approach, 1995, Pharmaceutical Biotechnology, Volume 6, Eds. Powell, M. F., and Newman, M. J., Plenum Press, New York and London, ISBN 0-306-44867-X). Preferred adjuvants for use with immunogens of the present invention include: aluminium or calcium salts (hydroxide or phosphate), oil in water emulsions (WO 95/17210, EP 0 399 843), or particulate carriers such as liposomes (WO 96/33739). Immunologically active saponin fractions (e.g. Quil A) having adjuvant activity derived from the bark of the South American tree Quillaja Saponaria Molina are particularly preferred. Derivatives of Quil A, for example QS21 (an HPLC purified fraction derivative of Quil A), and the method of its production is disclosed in U.S. Pat. No. 5,057,540. Amongst QS21 (known as QA21) other fractions such as QA17 are also disclosed. 3 De-O-acylated monophosphoryl lipid A (3D-MPL) is a well known adjuvant manufactured by Ribi Immunochem, Montana. It can be prepared by the methods taught in GB 2122204B. A preferred form of 3D-MPL is in the form of an emulsion wherein the 3D-MPL has a small particle size of less than 0.2 μm in diameter (EP 0 689 454 B1). Other non-toxic derivatives of Lipid A may also be used.

Adjuvants also include, but are not limited to, muramyl dipeptide and saponins such as Quil A, bacterial lipopolysaccharides such as 3D-MPL (3-O-deacylated monophosphoryl lipid A), or TDM. As a further exemplary alternative, the protein can be encapsulated within microparticles such as liposomes, or in non-particulate suspensions of polyoxyethylene ether (WO 99/52549). Particularly preferred adjuvants are combinations of 3D-MPL and QS21 (EP 0 671 948 B1), oil in water emulsions comprising 3D-MPL and QS21 (WO 95/17210, PCT/EP98/05714), 3D-MPL formulated with other carriers (EP 0 689 454 B 1), or QS21 formulated in cholesterol containing liposomes (WO 96/33739), or immunostimulatory oligonucleotides (WO 96/02555).

The vaccines of the present invention will be generally administered for both priming and boosting doses. It is expected that the boosting doses will be adequately spaced, or preferably given yearly or at such times where the levels of circulating antibody fall below a desired level. Boosting doses may consist of the peptide in the absence of the original carrier molecule (or Th epitope). Such booster constructs may comprise an alternative carrier (or Th epitope) or may be in the absence of any carrier (or Th epitope).

In a further aspect of the present invention there is provided a vaccine or immunogenic composition as herein described for use in medicine.

The immunogenic composition or vaccine preparations of the present invention may be used to protect or treat a mammal susceptible to, or suffering from dyslipidaemia or atherosclerosis, by means of administering said vaccine via systemic or mucosal route. These administrations may include injection via the intramuscular, intraperitoneal, intradermal or subcutaneous routes; or via mucosal administration to the oral/alimentary, respiratory, genitourinary tracts.

The amount of protein in each vaccine or immunogenic composition dose is selected as an amount which induces an immunoprotective response without significant, adverse side effects in typical vaccinees. Such amount will vary depending upon which specific immunogen is employed and how it is presented. Generally, it is expected that each dose will comprise 1-1000 μg of protein, preferably 1-500 μg, preferably 1-100 μg, of which 1 to 50 μg is the most preferable range. An optimal amount for a particular vaccine can be ascertained by standard studies involving observation of appropriate immune responses in subjects. Following an initial vaccination, subjects may receive one or several booster immunisations adequately spaced.

Vaccine preparation is generally described in New Trends and Developments in Vaccines, edited by Voller et al., University Park Press, Baltimore, Md., U.S.A. 1978. Conjugation of proteins to macromolecules is disclosed by Likhite, U.S. Pat. No. 4,372,945 and by Armor et al., U.S. Pat. No. 4, 474,757.

Passive Immunotherapy

In a second aspect of the present invention are provided polyclonal and monoclonal antibodies capable of binding to epitopes generated by modification of ApoCIII. Further, antibodies are provided which are capable of binding to SEQ ID NO.s 1 to 47 (preferably SEQ ID NO: 2 or 3) in which at least one amino-acid of the ApoCIII peptide sequence is modified.

The term “antibody” herein is used to refer to a molecule having a useful antigen binding specificity. Those skilled in the art will readily appreciate that this term may also cover polypeptides which are fragments of or derivatives of antibodies yet which can show the same or a closely similar functionality. Such antibody fragments or derivatives are intended to be encompassed by the term antibody as used herein.

The term “monoclonal antibody” is used herein to encompass any isolated antibodies such as conventional monoclonal antibody hybridomas, but also to encompass isolated monospecific antibodies produced by any cell, such as for example a sample of identical human immunoglobulins expressed in a mammalian cell line.

The monoclonal antibodies of the present invention are capable of being used in passive prophylaxis or therapy, by administration of the antibodies into a patient, for the amelioration of atherogenic disease.

The monoclonal antibodies of the present invention may be generated using the immunogens of the present invention (using known techniques e.g. Kohler and Milstein, Nature, 1975, 256, p495).

Also, there is provided by the present invention, an isolated antibody generated against the immunogens of the present invention.

Hybridomas secreting the monoclonal antibody ligands of the present invention are also provided.

Pharmaceutical compositions comprising the ligands, described above, also form an aspect of the present invention. Also provided are the use of the ligands in medicine, and in the manufacture of medicaments for the treatment of atherosclerosis.

In the passive treatments of atherosclerosis as provided herein, the administration of the ligands or antibodies of the present invention will be administered (preferably intra-venously) to the patients in need thereof. The frequency of administration may be determined clinically by following the decline of antibody titres in the serum of patients over time, but in any event may be at a frequency of 1 to 52 times per year, and most preferably between 1 and 12 times per year. Quantities of antibody or ligand may vary according to the severity of the disease, or half-life of the antibody in the serum, but preferably will be in the range of 1 to 10 mg/kg of patient, and preferably within the range of 1 to 5 mg/kg of patient, and most preferably 1 to 2 mg/kg of patient.

The immunogens, immunogenic compositions, vaccines or monoclonal antibodies of the present invention may be administered to a patient who is suffering from, or is at risk to, atherosclerotic disease, and are effective in re-establishing the correct equilibrium of the “bad” lipoproteins to the “good” lipoproteins balance, and minimise the circulation time of oxidised lipoproteins.

The present invention, therefore, provides the use of the modified ApoCIII epitopes, ligands (monoclonal antibodies) and immunogens of the present invention (as defined above), in the manufacture of pharmaceutical compositions for the prophylaxis or therapy of atherosclerosis. Accordingly, the modified ApoCIII immunogens of the present invention are provided for use in medicine, and in the medical treatment or prophylaxis of atherosclerosis.

There is also provided a method of treatment or prophylaxis of atherosclerosis comprising the administration to a patient suffering from or susceptible to atherosclerosis, of an immunogenic composition or vaccine or ligand of the present invention.

A method of prophylaxis or treatment of atherosclerosis is provided which comprises a reduction of total circulating triglyceride levels in a patient, by the administration of a vaccine of the present invention to the patient. In particular there is provided a method of reducing the amount of circulating VLDL and LDL, preferably oxidised VLDL and LDL, in a patient, by the administration of the vaccine or ligands of the present invention to the patient.

Also provided is a method of prophylaxis or treatment of atherosclerosis by the administration to a patient of a vaccine which is capable of reducing the average circulation time of ApoB containing lipoproteins. In this regard the average circulation time of ApoB containing lipoproteins, may be investigated in an in vivo animal model by the measuring the clearance rate of labelled ApoB containing lipoproteins from the plasma of the mammal (half-life of labelled ApoB containing lipoproteins).

A preferred immunogen for these method of treatment aspects of the present invention comprises or contains the modified ApoCIII epitopes SEQ ID NO: 1-47 (preferably SEQ ID NO: 2 or 3), in which at least one amino-acid of the ApoCIII peptide sequence is modified.

Preferred methods of treating individuals suffering from Atherosclerosis having elevated levels of modified, preferably oxidised or glycoxylated, ApoCIII comprise reducing the levels of modified ApoCIII, by the administration of a vaccine comprising or containing the modified ApoCIII epitope SEQ ID NO: 1-47 in which at least one amino-acid of the ApoCIII peptide sequence is modified (preferably SEQ ID NO: 2 or 3), or mimotope thereof, as an immunogen to said individual. Alternatively, in a related aspect of the present invention there is provided a method of treatment or prophylaxis of atherosclerosis by reducing the levels of circulating ApoCIII in the plasma of a patient, by administration of a monoclonal Ab that is capable of blocking the activity of ApoCIII, by binding to the epitope SEQ ID NO: 1-47 in which at least one amino-acid of the ApoCIII peptide sequence is modified (preferably SEQ ID NO. 2 or 3) and thereby abrogating the ApoCIII-mediated inhibition of lipoprotein lipase and/or the binding of ApoB to its receptor, to said patient.

Also provided by the present invention is a method of treatment or prophylaxis of atherosclerosis by reducing the number of ApoCIII molecules which are associated with an ApoB molecule in situ in the context of a lipoprotein by administration of a monoclonal Ab, or vaccine of the present invention. In a normal individual there is approximately one ApoB present in an LDL particle, the ApoB being associated with between 1-5 ApoCIII molecules. In diseased individuals the number of ApoCIII molecules may increase to up to 25. Accordingly, there is provided by the present invention a method of treatment or prophylaxis of atherosclerosis by reducing the ratio of ApoCIII molecules per ApoB molecules in the LDL in an individual with atherosclerosis from a high disease state level (approximately 20 to 25: 1) to a reduced therapeutic level preferably below 15: 1, more preferably below 10:1 and more preferably below 5:1, preferably below 3:1, and most preferably approximately 1:1 ApoC:ApoB. Levels of ApoCIII contained within ApoB-containing lipoproteins may be measured by nephelometry or electro-immunodiffusion (normal range is 2 to 3 mg/dL)

Also provided by the present invention is a combination therapy for treatment or prophylaxis of atherosclerosis comprising the active or passive immunotherapy of the present invention in combination with any other therapy or combination of therapies for treatment or prophylaxis of atherosclerosis, such as immunotherapy directed towards modified ApoCIII, oxidised ApoA, oxidised ApoB (as described in WO02/080954), oxidised LDL (WO02/50550) or cholesterol ester transfer protein (CETP; WO99/15655), or other known therapies.

The present invention is illustrated, but not limited, by the following examples:

### EXAMPLES

### Example 1

### Peptide synthesis

The ApoCIII peptides (1-79, 12-21, 12-35, 45-65, 19-28, 26-35, 1-17, 17-24 and 45-76 were synthesised by the solid phase method (Merrifield, 1986) on an automated synthesiser Model ABI 433A (Applied Biosystems Inc.) using Boc/Bzl strategy on a Boc-Ala-PAM resin for total apo CIII and MBHA resin for the others fragments. Each amino acid was coupled twice by dicyclohexylcarbodiimide/hydroxybenzotriazole without capping. Side chain protecting groups were as follows: Arg(Ts), Asp(Ochex), Glu(Ochex), Lys(2-Cl-Z), His(Dnp), Ser(Bzl), Thr(Bzl), Met(O)and Tyr(Br-Z). According to the sequence, the group Dnp on His was removed from the peptide, prior to the cleavage from its support by treatment with 10% β-mercaptoethanol, 5% diisopropylethylamine in DCM for 2 h and in NMP for 2 h. The peptidyl resin was then treated with 50% TFA in DCM for 20 min to remove the amino-terminal Boc. The peptide was cleaved from the resin and simultaneously deprotected according to a low and high HF procedure: the resin (1 g) was treated with anhydrous HF (2.5 mL) in the presence of p-cresol (0.75 g), p-thiocresol (0.25 g) and dimethylsulfide (6.5 mL) at 0° C. After 3 h hydrogen fluoride and dimethylsulfide were removed by vacuum evaporation and the residual scavengers and by products were extracted with diethyl ether. The reaction vessel was recharged with p-cresol (0.75 g), p-thiocresol (0.25 g) and 10 ml of anhydrous HF and the mixture was allowed to react at 0° C. for 1.5 h. Hydrogen fluoride was removed by evaporation and the residue was triturated with diethyl ether. The residue was filtered off, washed with diethyl ether and extracted with 200 ml of 10% aqueous acetic acid and lyophilised. The crude product was analysed by reversed-phase HPLC on a Vydac C18 column (4,6×250 mm, 5 μ, 100 A) using 60 min linear gradient from 0 to 100% Buffer B (Buffer A: 0.05% TFA in H₂O and Buffer B: 0.05% TFA, 60% CH₃CN in H₂O) at flow rate of 0.7 ml/min and detection was performed at 215 nm. Synthetic peptide were purified by RP-HPLC and were characterised and analysed by HPLC, the molecular mass determined by spectrometry.

### Example 2

### Modification of Peptides

Peptides may be modified by oxidation, nitration or glycosylation according to methods known in the art. Examples are included below, but the invention is not limited to these examples.

Modification by Oxidation with Copper Ions

Synthetic peptides produced according to the method of Example 1, or isolated ApoCIII, may be oxidised by incubation with Cu(II) (cupric sulfate) in 50 mM potassium phosphate, pH 7.4, at 37° C., for 90 minutes (WO02/080954).

Modification by Oxidation Using MDA

Synthetic peptides produced according to the method of Example 1, or isolated ApoCIII, may be oxidised by incubation with 0.5M malone dealdehyde (MDA; Sigma) for 3h at 37° C., followed by dialysis against PBS-EDTA (phosphate-buffered saline (PBS) containing 1 mM EDTA) at 4° C.; oxidation of the peptides may be investigated by use of SDS-PAGE gels (WO02/080954).

Modification by Oxidation Using Myeloperoxidase (MPO)

Synthetic peptides produced according to the method of Example 1, or isolated ApoCIII, may be oxidised by use of myeloperoxidase as described in WO02/50550.

Modification by Oxidation Using Hypochlorite

Synthetic peptides produced according to the method of Example 1, or isolated ApoCIII, may be oxidised with hypochlorite (HOCI). Oxidation with reagent HOCl should be carried out on ice, at a pH of 7.4, by adding 1 volume of reagent HOCl, freshly diluted in phosphate buffer (50 mM), to 4 volumes of peptide (concentration: around 2 mg of protein/ml). The peptides may then be exposed to reagent HOCl in ratios of around 0.06 to 0.88 mol of HOCl per mol of amino acid residue. After addition of HOCl, the reaction mixture is mixed briefly (<1 s) and left on ice for 15 min, whilst being protected from light.

Modification by Nitration

Synthetic peptides produced according to the method of Example 1, or isolated ApoCIII, may be nitrated as follows: after dialysis against 0.05 M PBS, pH 7.5, containing 1.5 mM EDTA, the peptides are nitrated by three additions of a peroxynitrite solution in Tris-HCl buffer, pH 9.0, containing 1.5 mM EDTA, at 4° C. Nitration may be stopped by dialysis against PBS-EDTA.

Modification by Glycosylation

Synthetic peptides produced according to the method of Example 1, or isolated ApoCIII, may be glycosylated by incubation with 80 mmol/l glucose in PBS containing 2 mmol/l EDTA, 1,000 UI/100 ml aprotinin, 100 μM deferoxamine, and 0.009% gentamicin for 10 days at 37° C.

Modification by Glycoxylation

Synthetic peptides produced according to the method of Example 1, or isolated ApoCIII, which have been glycosylated, may then be oxidised by any of the three methods described herein to form glycoxylated ApoCIII.

### Example 3

### Monoclonal Antibody Production

Monoclonal antibodies may be prepared according to the following method: Peptides may be synthesised as described in Example 1 with the addition of a small linker sequence (CGG) onto the carboxyl end of the peptide. The conjugate may be produced using maleimide chemistry, by reacting this modified sequence with a commercial pre-activated BSA. BSA may be purchased from Pierce, and pre-activated with a succinimidyl 4-(N-maleimidomethyl)-cyclohexane-1-carboxylate (SMCC) linker. SMCC may also be bought from any major manufacturer and used following the manufacturers instructions. The coupling of BSA to the peptide via the SMCC may be carried out over 2 hr at room temperature with an excess of peptide, before quenching with the reaction with excess cysteine, followed by dialysis against phosphate buffer.

A group of BalbC mice may be immunised with 25 μg of conjugate BSA-peptide 12-35 formulated in an oil in water emulsion described in WO 95/17210 andntra muscular injections done at day 0, 14, 28.

Sera from the mice may be evaluated by ELISA for strongest anti-peptide and anti-complete modified ApoCIII responses.

Another functional assay may be performed by ELISA to identify the mouse with the highest anti-modified ApoCIII titres when modified ApoCIII is in its native form and loaded into the lipoproteins. Briefly, plates may be coated with affinity purified polyclonal antibodies to human modified ApoCIII. Plasma sample, HDL and VLDL particles may be incubated, and after washing, revealed by sera of the immunized mice.

After a two-month resting period, the “best” mouse may be boosted with antigen in saline and sacrificed three days later. Spleen cells may be fused with the Sp2/0 B cell line according to standard protocols. First screening of hybridoma supernatants may be performed by ELISA and positive wells subcloned and tested, also for reactivity against complete modified ApoCIII.

### Example 4

### Characterisation of the Antibodies

Peptide specificity ELISA.: Microtiter plates (flat-bottom 96-well EIA; Costar, Dutscher) may be washed with 0.1 mol/L phosphate-buffered saline (PBS, pH 7,2) before being coated with 100 μl/well of free peptide (5 μg/ml) and incubated overnight at room temperature. The plates may be washed four times with buffer and to minimise the non-specific binding to the microtiter wells, the plates may be saturated with 250 μL/well of bovine serum albumin at 3% in 0.1 M PBS buffer and incubated for 1 h at 37° C. The plates may be washed four times again and incubated for 2 h at 37° C. with 100 μL of the 35 monoclonal antibodies produced in example 2, diluted in 1% of bovine serum albumin in 0.1 M PBS buffer, then washed three four times with PBS. To assess the immunological reaction, 100 μL of 10 000-fold diluted, anti-mouse IgG labelled with peroxidase, in 0.1% of BSA in PBS buffer may be added to each well. After an incubation for 2 h at 37° C., the plates may be washed four times with PBS and 100 μL of substrate solution was added. The substrate solution may be prepared as follows: 30 mg of o-Phenylenediamine dihydrochloride may be dissolved in 20 ml of 0.1 mmol/L phosphate-citrate buffer, pH 5.5 containing 20 μL of 30% hydrogen peroxide. After 30 min at room temperature in the dark, the reaction may be stopped by adding to each well 100 μl of 1 mmol/L HCl. The absorbance may be measured at 492 nm.

Functional Assays

The objective is to check if the epitopes recognised by the monoclonal antibodies are accessible when ApoCIII is in the context of human plasma-purified lipoproteins (HDL, VLDL).

Sandwich ELISA: Microtiter plates (flat-bottom 96-well EIA; Costar, Dutscher) may be washed with 0.1 mol/L phosphate-buffered saline (PBS, pH 7.2) before being coated with 100 μL/well of polyclonal anti-ApoCIII, and incubated overnight at room temperature. The plates may be then washed four times with buffer and incubated for 2 h at 37° C. with 100 μL of dilutions of a sample (eg human plasma, purified human HDL, purified human VLDL, purified human LDL). To minimise the non-specific binding to the microtiter wells, the dilutions of antigen may be performed in 1% of bovine serum albumin in 0.1 M PBS buffer. 100 μL of the monoclonal antibodies from example 2 may be added and incubated for 2 hours at 37° C., and the immunological reaction detected as earlier described with anti-mouse peroxidase antibodies.

1. A peptide comprising isolated modified ApoCIII peptide having a primary sequence of SEQ ID NO. 1 or fragment thereof in which at least one amino-acid of the ApoCIII peptide sequence is modified, or a mimotope thereof.

2. The peptide of claim 1 comprising the epitope described in SEQ ID NO. 2 or fragment thereof in which at least one amino-acid of the ApoCIII peptide sequence is modified, or mimotope thereof.

3. The peptide of claim 1 comprising the epitope described in SEQ ID NO. 3 or fragment thereof in which at least one amino-acid of the ApoCIII peptide sequence is modified, or mimotope thereof.

4. The peptide of claim 1 comprising the epitope described in SEQ ID NO. 447 or fragment thereof in which at least one amino-acid of the ApoCIII peptide sequence is modified, or mimotope thereof.

5. A peptide according to claim 1 in which the at least one amino-acid is modified by one or more of oxidation, nitration, glycosylation, hydrogenation.

6. A peptide according to claim 1 in which the at least one amino-acid is modified by oxidation, or a fragment thereof.

7. A peptide according to clam 1 in which the at least one amino-acid is modified by glycoxidation, or a fragment thereof.

8. A peptide according to claim 5 in which the at least one amino acid is oxidised or glycoxylated and in which an epitope present in circulating oxLDL is formed.

9. A vaccine immunogen comprising a peptide as claimed in claim 1, conjugated or fused to a carrier molecule.

10. A vaccine immunogen according to claim 9, in which the carrier molecule comprises a T-helper epitope.

11. A vaccine immunogen according to claim 9, in which the carrier molecule is tetanus toxoid, or a peptide of tetanus toxin.

12. A vaccine immunogen according to claim 9, in which the carrier molecule is P2.

13. A vaccine immunogen according to claim 9, in which the carrier molecule is P30.

14. A vaccine immunogen according to claim 9 in which the link between the carrier and the peptide is through the C-terminus of the peptide.

15. A vaccine immunogen according to claim 9 in which the link between the carrier and the peptide is through the N-terminus of the peptide.

16. A vaccine immunogen according to claim 9 in which the carrier is linked to two or more peptides.

17. A vaccine immunogen according to claim 9 in which the carrier is recombinantly fused between two peptides.

18. A vaccine immunogen according to claim 9 comprising n+l peptides and n carriers, in which the peptides and carriers are alternately fused to each other, and in which n=1,2,3,4,5, 6 or 7.

19. A vaccine immunogen according to claim 18 in which all the peptides have the same sequence.

20. A vaccine immunogen according to claim 9 comprising isolated ApoCIII (SEQ ID NO 1) conjugated or fused to P2 (peptide of TT) at the C-terminus of ApoCIII.

21. A vaccine immunogen according to claim 9 comprising isolated ApoCIII (SEQ ID NO 1) conjugated or fused to P2 (peptide of TT) at the N-terminus of ApoCIII.

22. A vaccine immunogen according to claim 9 comprising P2 fused between two peptides of SEQ ID NO 2 of ApoCIII

23. A vaccine immunogen according to claim 9 comprising P30 (peptide of TT) fused between two peptides of SEQ ID NO 2 of ApoCIII

24. A vaccine immunogen according to claim 9 comprising P2 fused between two peptides of SEQ ID NO 5 of ApoCIII

25. A vaccine immunogen according to claim 9 comprising P2 fused between two peptides of SEQ ID NO 4 of ApoCIII

26. A vaccine comprising a vaccine immunogen as claimed in claim 9, a pharmaceutically acceptable excipient, and optionally an adjuvant.

27. An isolated antibody elicited by the peptides as claimed in any one of claims 1-5

28. An isolated antibody elicited by the vaccine immunogen of claim 9.

29. A monoclonal antibody that is specific for the peptides as claimed in any one of claims 1-5.

30. A monoclonal antibody that is capable of competing with those monoclonal antibodies claimed in claim 26, for binding to human ApoCIII.

31. A process for oxidising ApoCIII or a fragment thereof comprising the sequence of SEQ ID NO 2-47, which optionally is glycosylated, by one or more of the following methods:
(a) oxidation with MPO
(b) oxidation with MDA
(c) oxidation with Copper ions
(d) oxidation with hypochlorite

32. A process according to claim 31 in which the oxidised ApoCIII or fragment thereof is conjugated to a carrier.

33. Oxidised ApoCIII, or a fragment thereof, produced according to the process of claim 3.

34. A vaccine comprising modified ApoCIII of claim 6, 7 or 31, a pharmaceutically acceptable excipient, and optionally an adjuvant, together with one or more of oxApoB, oxApoA, or CETP, or fragments thereof.

35. A method of treatment or prophylaxis of atherosclerosis of an individual in need thereof, by administration of a vaccine immunogen as claimed in claim 9 to said individual.

36. A method of treatment or prophylaxis of atherosclerosis of an individual in need thereof, by administration of a vaccine as claimed in claim 26 to said individual.

37. A method of treatment or prophylaxis of atherosclerosis of an individual in need thereof, by administration of a monoclonal antibody as claimed in claim 29 to said individual.

38. A method of treatment or prophylaxis of atherosclerosis of an individual in need thereof, by administration of a monoclonal antibody as claimed in claim 30 to said individual.

39. Use of a peptide as claimed in any one of claims 1-5 in the manufacture of a medicament for the prevention or treatment of atherosclerosis.

40. Use of a monoclonal antibody as claimed in claim 29 or 30 in the manufacture of a medicament for the prevention or treatment of atherosclerosis.
